(19) United States
(12) Patent Application Publication
(10) Pub. No.: US 2007/0129602 A1
(43) Pub. Date: Jun. 7, 2007

(54) Device, method and system for activating an in-vivo imaging device

(75) Inventors: Ido Bettesh, Haifa (IL); Jerome Avron, Haifa (IL)

Correspondence Address: PEARL COHEN ZEDEK LATZER, LLP, 1500 BROADWAY 12TH FLOOR, NEW YORK, NY, 10036, US

(73) Assignee: GIVEN IMAGING LTD.

(21) Appl. No.: 11/283,867

(22) Filed: Nov. 22, 2005

(51) Int. Cl. A61B 1/00; A61B 1/04

(52) U.S. Cl. 600/118; 600/109; 600/160

(57) ABSTRACT

A device, system, and method for activating an ingestible imaging device with an RF radiation signal such that an imaging device that may be initially in a dormant state may be activated prior to ingestion by exposure to RF radiation The device may include an RF switch that may facilitate powering of one or more electrical components of the device when toggled. RF switch may also serve deactivate the ingestible imaging device.

### FIELD OF THE INVENTION

The present invention relates to ingestible imaging devices, and more particularly to a device for activating an ingestible imaging device using radio frequency radiation

### BACKGROUND OF THE INVENTION

In-vivo sensing devices such as for example ingestible imaging capsules may include an autonomous power source such as for example a battery whose power may last for a limited period of time in use. To conserve power, it may be preferable to turn on the device very soon before the device may be ingested or swallowed Typically, the battery and all other components may be sealed in the device during manufacturing to insure for example durability and water-tightness of the in-vivo device. Such a device may not accommodate a manual or externally accessible switch or mechanism that may operate the device after it is sealed. Quality control standards may require that each device be tested prior to its use, which may require that the device be activated and deactivated possibly several times for testing purposes prior to an in-vivo operation.

Known in-vivo imaging device may include reed switches to activate the device prior to use. Reed switches may be sensitive to electromagnetic (EM) fields and may either close or open when exposed to an EM field of a predefined strength In some cases, known reed switches may be sensitive to mechanical shock, for example, during delivery and handling of imaging devices from the manufacturers to the customers In other cases, the reed switches may be sensitive to EM interference from the surrounding environment. In yet other cases, reed switches may suffer from a known stacking effect that may at times not be releasable under exposure of the EM field.

### SUMMARY OF THE INVENTION

According to embodiments of the present invention, there is provided a device, method, and system for activating an ingestible imaging device remotely by Radio Frequency (RF) radiation. In one example, activation may be facilitated with electrical components, e.g. mechanically static components According to one embodiment of the present invention, an RF operating switch contained within the ingestible device may change the operational state, e.g. alter the power state of the device when exposed to a predefined RF radiation signal. For example, the RF operating switch may wakeup the device from a dormant state, for example by supplying power, for example battery power, to one or more electrical components contained within the device. During an activated state, the ingestible imaging device may transmit image data and other data wirelessly from in-vivo to an external receiving device and/or may receive data, e g. control data. In another example, the RF operation switch may deactivate the device and return the device to a dormant state. In one example, activation and deactivation may be performed repeatedly according to need. According to one embodiment of the present invention, the change in the operational state may be retained subsequent to the termination of the RF radiation. Activation and deactivation of the device may be performed prior to ingesting the device.

### BRIEF DESCRIPTION OF THE DRAWINGS

The present invention will be understood and appreciated more fully from the following detailed description taken in conjunction with the drawings in which:

FIG. 1 is a simplified conceptual illustration of an in-vivo imaging system with an external RF radiation source according to an embodiment of the present invention;

FIG. 2 is a simplified diagram of an external RF radiation source according to an embodiment of the present invention;

FIG. 3 is a simplified circuit diagram showing an exemplary circuit diagram of an external RF radiation source according to an embodiment of the present invention;

FIG. 4 is a simplified circuit diagram showing an exemplary circuit diagram of an RF switch within an in-vivo device according to an embodiment of the present invention; and

FIG. 5 is a flow chart of a method of activating an ingestible device in accordance with an embodiment of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough under standing of the present invention. However, it will also be apparent to one skilled in the alt that the present invention may be practiced without the specific details presented herein. Furthermore, well-known features may be omitted or simplified in order not to obscure the present invention.

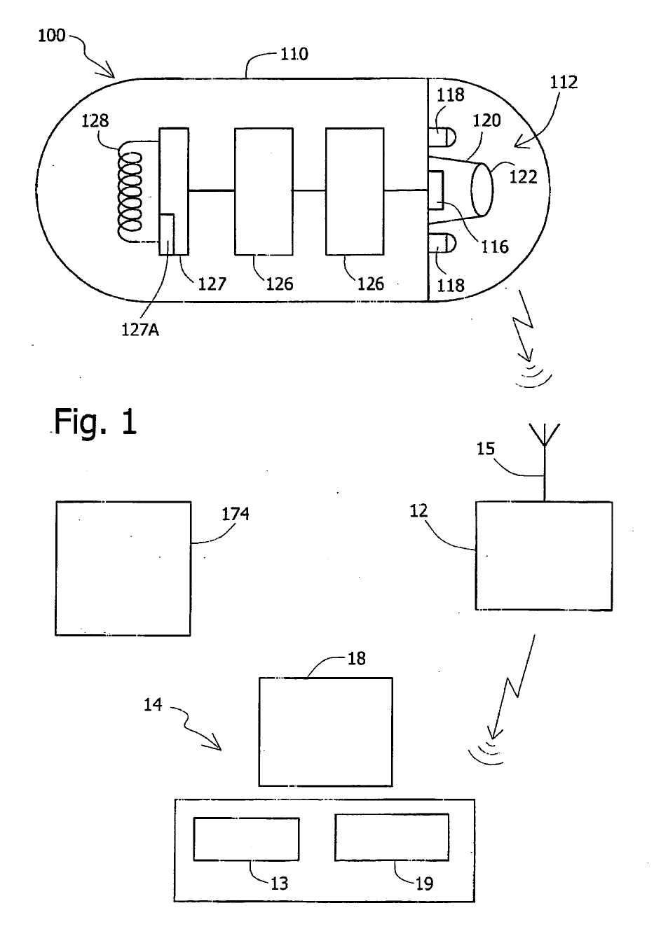

Reference is made to FIG. 1, showing a simplified conceptual illustration of an in-vivo imaging system with a remote RF radiation source according to an embodiment of the present invention. Device 100 may be an autonomous in-vivo sensor, for example, an in-vivo imaging device for gathering data in-vivo. An RF radiation source 174 may remotely activate device 100 from a dormant state prior to inserting device 100 in-vivo. When activated, data may be gathered in-vivo and may be transmitted to an external receiver 12, for example with an RF receiver having one or more receiving antennas 15. In some embodiments, receiver 12 may include a recorder and storage unit to record and store received data and may include processing capabilities. Data captured by device 100 and received by receiver 12 may be, for example downloaded to workstation 14 for processing, analysis, and display, for example in display unit 18. In one embodiment of the present invention, receiver 12 and workstation 14 may be integrated into a single unit, for example, may be integrated into a single portable unit. In other embodiments receiver 12 may be capable of transmitting signals to device 100 as well as receiving. In yet another embodiment of the present invention, receiver 12 may include display capability, for example receiver 12 may include an online viewer.

Device 100 may include a sensing device such as for example an imaging unit 112 within an outer covering or housing 110, constructed and operative in accordance with an embodiment of the invention. Housing 110 may be, for example, spherical, ovoid, or any other suitable shape and may be partially deformable. Imaging unit 112 may typically include at least one imager 116, which may be or may include a charge coupled device (CCD), a complementary metal oxide semiconductor (CMOS) imager, another suitable solid-state imager or other imagers. In addition imaging unit 112 may include, for example a lens 122 and a lens holder 120 as well as one or more (e.g., a pair, a ring, etc.) illumination sources 118, such as for example, light emitting diodes (LEDs), which may illuminate the areas to be imaged by the imager 116. Other positions for image 116, illumination sources 118 and other components may be used and other shapes of a housing 110 may be used.

Device 100 may include and/or contain one or more power units 126, a transmitter 127, e.g. an RF transmitter, and one or more antennas 128 for transmitting and/or receiving data, e.g. receiving control data. Power unit 126 may include one or more batteries and/or other suitable power sources. In another example power unit 126 may include a power induction unit that may receive power from an external source. In one example, transmitter 127 may include control capability, for example transmitter 127 may be or include a controller for controlling various operations of device 100, although control capability or one or more aspects of control may be included in a separate component such as for example circuit board or other circuitry included in device 100. Transmitter 127 may typically be included on an Application Specific Integrated Circuit (ASIC), but may be of other constructions. Device 100 may include a processing unit separate from transmitter 127 that may, for example, contain or process instructions. Transmitter 127 may at least partially include the components of an RF operating switch 127a that may control activation of device 100, for example powering of transmitter 127, imager 116 and illumination source 118. Other components may be activated directly or indirectly by RF operating switch 127a. RF switch 127a may function as an operating switch to activate and/or deactivate and/or control device 100 or components of device 100 on demand. In one example, one or more low power components of device 100 may be powered during the dormant stage of device 100 to facilitate waking up of the transmitter on demand. According to one embodiment, it may be desirable to maintain device 100 in a dormant state prior to use so as not to deplete power source 126. During a dormant state, device 100 may consume minimal power.

Device 100 may be inserted in-vivo, for example by swallowing or ingesting. Device 100 may enter a body lumen for in-vivo imaging and may be, for example, fixed at a position in the body or it may move through for example a GI tract or other body lumen. Device 100 may include components and operate similarly to the imaging systems described in U.S. Pat. No. 5,604,531 to Iddan, et al., in U.S. Patent Application Publication Number 20010035902, entitled “Device and system for in vivo imaging”, published on Nov. 1, 2001 and/or U.S. Patent Application Publication Number 20020109774, entitled “System and method for wide field imaging of body lumens”, published on Aug. 15, 2002, each assigned to the common assignee of the present application and each hereby fully incorporated by reference. Furthermore, a reception, processing and review system may be used, such as in accordance with embodiments of U.S. Pat. No. 5,604,531, U.S. Patent Application Publication Number 20010035902, and/or U.S. Patent Application Publication Number 2002-0109774, although other suitable reception, processing and review systems may be used.

In an embodiment, components of device may be sealed, e.g. water tightly sealed, within the housing 110 and the body or shell may include more than one piece. For example, an imager 116, illumination sources, power source 126, transmitter 127, and circuit board 124, may be sealed and or contained within the device body.

Device 100 may be a capsule or other unit that does not require wires or cables external to device 100, for example, to receive power or transmit information. For example, power may be provided by an internal battery. Other embodiments may have other configurations and capabilities. For example, components may be distributed over multiple sites or units. Control information may be received from an external source. Device 100 may initially be in a dormant state and then be activated and/or woken up prior to ingesting by exposing device 100 to a predefined level and/or pattern of RF radiation. RF radiation may induce energy in antenna 128 and transmit a signal to RF switch 127a to activate operation of device 100. In an alternate embodiment, RF switch 127a may also serve to deactivate operation of device 100. An external RF radiation source 174 may be used to generate the required RF radiation signal to operate, turn on, or wake up device 100. According to one embodiment of the present invention, the generated signal may have a predetermined pattern. For example, a first defined pattern may give indication to RF switch 127a to wake device 100 up and a second defined pattern may give indication to RF switch 127a to return device 100 to a dormant state The number of activations and deactivations of device 100 may be unlimited Other signals may be implemented to control the operational state, e.g. the power state, or the function state of device 100.

In one embodiment, since electrical components are sealed or otherwise contained within a housing or shell, the device or components of the device may be activated, turned on, or deactivated or turned off using, for example a remote signal, such as an RF signal, generated outside the device.

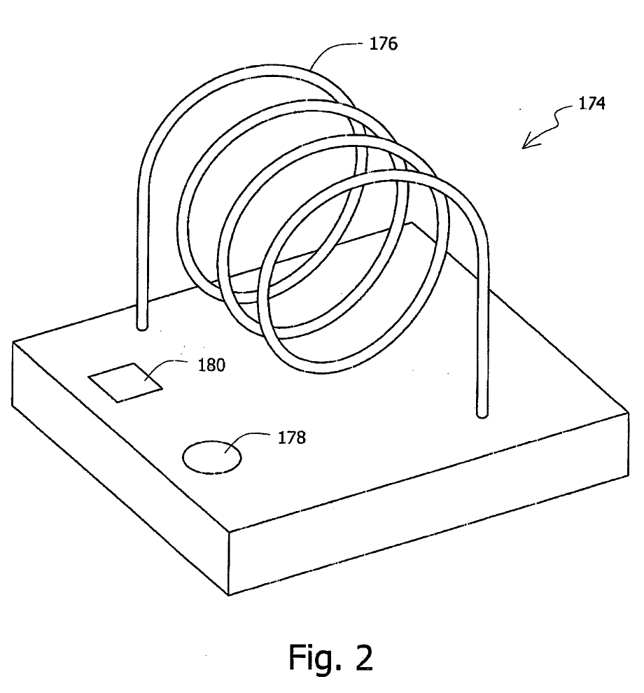

Reference is made to FIG. 2 showing a simplified diagram of an external RF radiation source 174 that may be used to generate an RF signal to activate and/or deactivate device 100 prior to ingesting device 100 and/or prior to inserting the device 100 in-vivo according to an embodiment of the present invention. In one example, RF radiation source 174 may generate RF radiation signal to wake up device 100 from a dormant state. According to one embodiment of the present invention, an electrically powered coil may generate an RF radiation signal when current flows through coil 176. Current flow may be initiated by activating the unit's controller and/or operating switch 178, for example, a button or dial switch. Other methods of control may be used. Device 100 may be inserted into activated coil 176 of RF radiation source 174 for activation, for example inserted such that internal coil 128 of device 100 may be substantially parallel or co-linear with coil 176. An RF switch 127a within device 100 upon activation may retain the device in an operationally active state subsequent to the activation or may maintain that state until an additional and/or alternate RF radiation signal, e.g. RF radiation pattern may be received by RF switch 127a. For example, the power supplied to one of more electrical components of device 100 through RF switch 127a may be supplied subsequent to termination of the radio frequency radiation. Subsequently to activating device 100, device 100 may be inserted in-vivo for capturing and transmitting in-vivo data through one or more in-vivo body lumens.

In one embodiment, coil 176 may additionally be used to sense the operating status of a device 100 inserted within coil 176. For example, coil 176 or another component of RF radiation source 174 may also act as a receiving antenna that may pick up signals transmitted by device 100, e.g. signals indicating the operational status of device 100. Other signals may be picked up and for example, processed to indicate, for example, an operational status of device 100. A status LED 180 may indicate the sensed operating status of device 100. For example, a green light may be lit when device 100 may be in an operationally activated state. A red light may be lit when device 100 may be in an operationally dormant state. Other suitable indications may be made. Controller 178 may be used to toggle device from an operationally dormant state to an operationally active state and visa versa.

In one embodiment of the present invention, RF radiation source may be a stand alone unit, may be portable or suitable for placement on a desk top. In other embodiments of the present invention, RF radiation source 174 may be integral to receiver 12 and/or workstation 14 and may take other suitable forms. In another example, RF radiation source 174 may be integral to the packaging of device 100, for example the blister packaging for device 100, e.g. opening of the blister may initiate operation of RF radiation source 174. Other configurations are possible. In some embodiments, unit 174 may include an independent or portable power source such as for example a battery. In some embodiments, unit 174 may issue a signal such as a buzz or beep to indicate that a device has been turned on or activated. In some embodiments, unit 174 may evaluate the functions of a device that is activated to determine whether the activated device is operating properly and/or as desired. For example, unit 174 may evaluate whether a battery or power source inside an ingestible sensor is operating. Other features or components may be included in unit 174, and other configurations are possible. Other methods of generating radio frequency radiation to device 100 may be possible. Operating device may act as a transformer, transferring energy, for example in the RF range to an antenna 128 of device 100 and thereby activating and/or deactivating an RF switch 127a.

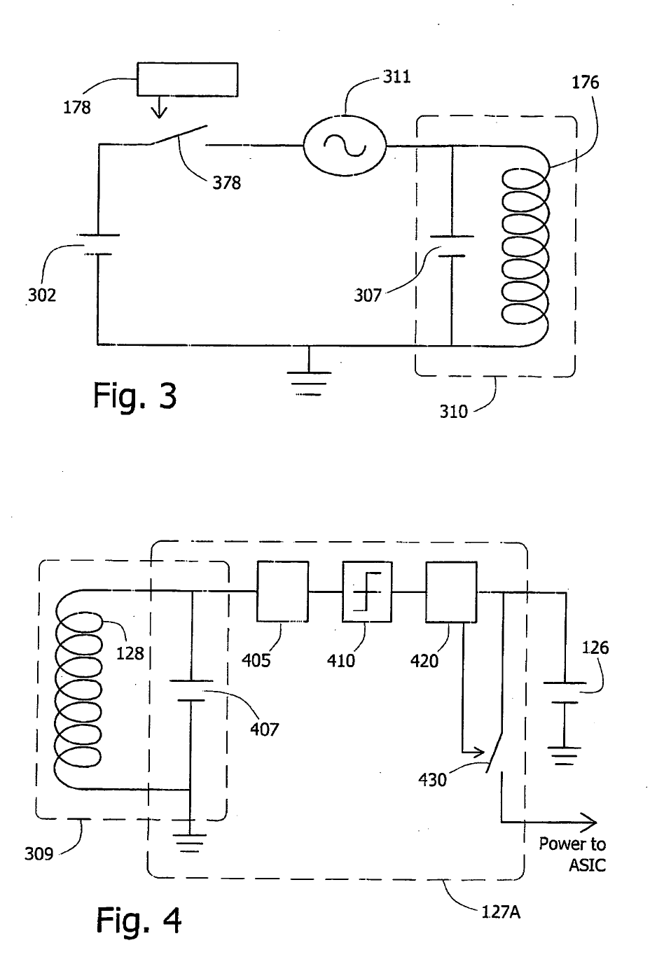

Reference is now made to FIG. 3 showing a simplified circuit diagram of an RF radiation source 174 according to an embodiment of the present invention. RF radiation source 174 may include a power source 302, for example, a DC power source, e.g. a battery that may power RF radiation source 174 and a controller 178 to control the operation of unit 174. In one example, controller 178 may control an internal switch 378. In another example controller 178 and switch 378 may be one in the same, e.g may be a single component. According to one embodiment of the present invention, RF radiation source 174 may operate in frequencies that may be typical to frequencies used to operate an RFID tag. For example, typical frequencies may include 13.56 MHz, 27.12 MHz, 865 MHz, and 2.45 GHz. Other RFID frequencies or other suitable frequencies may be used. Oscillator 311 may, when powered, generate a desired current to the parallel resonance circuit 310 that may include for example coil 176 and capacitor 307. The parallel resonance circuit 310 may be tuned, for example, to have the same resonance frequency as resonance circuit 309 (FIG. 4). The resonance circuit 310 may amplify the current from oscillator 311, for example by a Quality (Q) factor. In one example, the Q factor may range, for example between the ranges of 10-100. Other suitable ranges may be used.

In other embodiments, the parallel resonance circuit 310 may be replaced by a serial resonance circuit. Circuitry of unit 174 may be similar to a transformer where coil 176 may be a primary coil that may induce voltage to a secondary coil, for example, antenna 128 within device 100. Other suitable circuitries may be used to generate an RF radiation signal to operationally activate a device 100. According to one embodiment of the present invention, coil 176 and antenna 128 may be placed in a position relative to each other such that the maximum and/or sufficient amount voltage and/or current may be induced from antenna 176 to antenna 128. According to one embodiment of the present invention, antennas 176 and 128 may be coils and the desired relative position maybe such that antennas 176 and 128 may be parallel and/or collinear with respect to each other.

In some embodiments, when device 100 is placed substantially within coil 176, the RF radiation generated within the coil may induce voltage in antenna 128 within device 100 to a level that may activate RF switch 127a within device 100.

Reference is now made to FIG. 4 showing an exemplary circuit diagram of an RF switch within an in-vivo device 100 according to an embodiment of the present invention. Other suitable switches or devices receiving RF energy and acting as a switch or controller may be used. Energy radiated by coil 176 may induce voltage on antenna 128 of device 100 which may trigger the RF switch 127a to for example change the power state of device 100. According to one embodiment of the present invention, a rectifying circuit 405, for example an AC to DC converter, may be implemented to rectify the signal received, for example a diode bridge and capacitor. A threshold block 410 may perform thresholding so that only signals above a defined threshold may initiate activation of device 100. For example, a signal above a defined threshold may signal a controller 420 to, for example, change the position of switch 430 to, for example, power, e.g. with power source 126, and/or wake up transmitter 127. Other components may be powered with RF switch 127a. In one example, a signal of amplitude 1 volt may be required to pass threshold block 410. Other amplitude levels may be used. Controller 420, rectifying circuit 405, threshold block 410, or other components, or their functionalities, may be included within, for example, transmitter 127.

According to one embodiment of the present invention, the RF signal generated by device 174 may generate an electromagnetic field in the range between 1 to 100 microWB/mˆ2. Other ranges of electromagnetic fields may be generated. In other embodiments of the present invention, in order to avoid false activation and/or deactivation of device 100, a pre-defined number of pulses or some pattern of a signal may need to pass threshold block 410 before the device may change its operating state In one embodiment of the present invention, controller 420 may include a timer and counters as well as other components or circuitries. A timer may, for example, be used to measure time intervals between pulses that may pass threshold 410. Counters may be used to count the number of pulses. For example, in order to activate device 100, four pulses may be required to pass the threshold 410 and the time interval between a pair of the pulses that pass the threshold 410 may be required to be within a time range, e.g. 5 to 10 msec. Other numbers of pulses may be used. According to one embodiment of the present invention, one or more timers may be activated only after a first pulse may have passed the threshold 410 hence saving power during a dormant state. Other methods of detecting an activation signal may be implemented.

During the dormant state of device 100, controller 420 may be powered by power source 126 so that operational activation of device 100 may be accomplished. The power consumption of controller 420 may be minimal during a dormant state, for example, between 50 to 200 nonoAmp, e.g. 100 nanoAmp or 200 nanoAmp, so as not to deplete the power source 126 of device 100. In one example controller 420 may be only partially activated during a dormant state to facilitate minimal power consumption.

In an alternate embodiment of the present invention, RF switch may be a toggle switch that may deactivate device 100 in a similar manner used to activate device 100. For example, a first pulse and/or set of pulses may activate device 100 and subsequent pulse or set of pulses may serve to deactivate device 100. In another embodiment a first pattern of pulses may be used and/or required to activate device 100 and a second pattern of pulses may be used and/or required to deactivate device 100. Other methods of altering the power state of device 100, e.g. activating and/or deactivation device 100 may be implemented.

In some embodiments, switch 127a may be toggled into a fixed or permanently closed position such that switch 127a may retain a closed position or ‘on state’ even after the RF field created by the external RF radiation source 174 may have ceased and/or terminated.

In operation, device 100 may be manufactured, packaged, or shipped with switch 127a in an open position such that some or all of power from, for example power source 126 is not supplied to the electrical components (e.g. illumination source 118, transmitter 127, imager 116, etc.) of the device 100, and so that one or more functions of device 100, such as for example the imager function, is dormant or not operative. At a desired time, such as for example when device 100 is to be tested or before device 100 is to be ingested by a patient or user, antenna 128 may be exposed to radio frequency radiation generated by external RF radiation source 174 while device 100 is still outside a body, or ex-vivo. As a result of the induced voltage on antenna 128, RF switch 127a may toggle into a closed position. The closed switch may allow power from, for example power source 126 to power one or more electrical components of device 100.

In some embodiments, a further exposure of antenna 128 to a pre-defined RF radiation may toggle the RF switch to an open position, thereby shutting off a power supply of device 100 and de-activating one or more functions and/or electrical components of device 100. In some embodiments, the activation and deactivation capability of device 100 may be used during testing device 100, such as for example factory testing prior to shipment. Activation and deactivation of device 100 may be performed repeatedly as required.

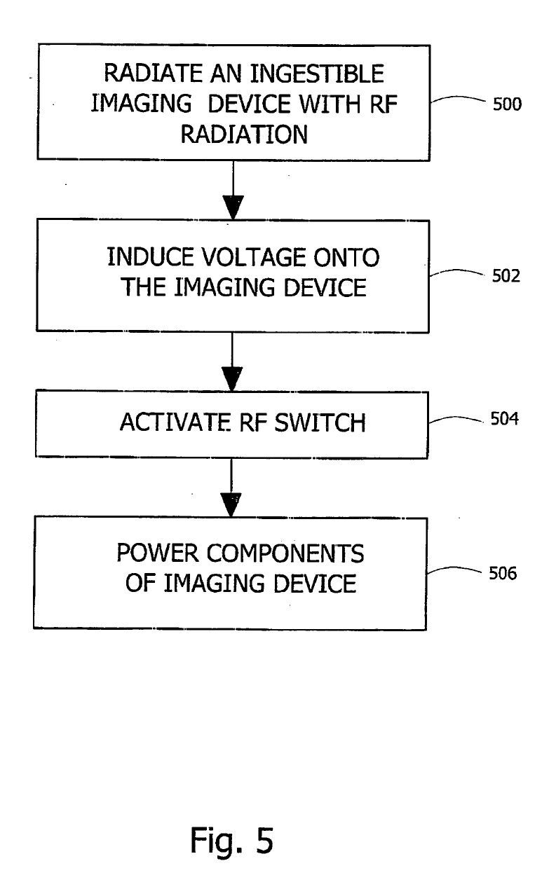

Reference is made to FIG. 5, a flow chart of a method of activating an ingestible sensor in accordance with an embodiment of the invention. In block 500, an ingestible imaging device may be radiated with RF radiation from an external source. For example, a device 100 such as for example an ingestible imaging device may be placed in proximity to or within a coil 176 generating radio frequency radiation. In some embodiments, inserting the device 100 into, for example, a strong RF electromagnetic field may be implemented by inserting the device 100 substantially within a coil 176. Coil 176 may yield a substantially strong electromagnetic field. In some embodiments, such placing may be performed ex-vivo, or prior to the ingestion of the device or the insertion of the device into an in-vivo area. In some embodiments the radio frequency radiation may be at a pre-determined power and/or frequency. In some embodiments, the generator of RF radiation may be suitable for generating radiation between 1 to 100 micro WB/mˆ2, for example 1.5 micro WB/mˆ2, for a period of several seconds.

In block 502, energy from RF radiation may induce voltage onto a component within an ingestible imaging device. In some embodiments, such energy may be collected by, for example, coil 128 which may be in resonance with capacitor 407. In block 504, when the induced voltage is sufficiently high, a switch may be activated or toggled, and a position of such switch may go from on to off, or for example off to on. In one embodiment of the present invention a signal to wake up device 100, or to turn on components in device 100 may be a predefined signal, for example a predefined pattern of RF pulses, while a signal to turn device 100, or components in device 100 off may be an alternate predefined signal, e.g. a second predefined pattern of RF pulses. In one example, the RF signal required to turn device 100 or its components off may be a simpler and/or shorter signal, e.g. a shorter predefined pattern of pulses compared with the signal that may be required and/or predefined to wakeup device 100. In other embodiments different signal patterns may be defined to control operation of device 100.

In block 506, power from power source 126 may be supplied to one or more electrical component of device 100. In some embodiments, the activation of a switch 430 may close a circuit that may include one or more electrical components (e.g. transmitter 127, illumination source 118, imager 116, or other components). In some embodiments, the switch may permanently close such circuit so that such switch is fixed in an on position, and so that power may continue to flow from a power source to a component of the device 100 even after RF radiation has ceased. Other methods of activating, by remote control an ingestible device with the use of RF radiation may be implemented.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the true spirit and scope of the invention.

1. An ingestible imaging device comprising:
an imager;
a power source; and
an operating switch, wherein the operating switch is activated by radio frequency radiation prior to ingestion.

2. The device according to claim 1 comprising an electrical component, wherein the operating switch is to supply power from a power source to the electrical component, and wherein the power source and the electrical component are contained within the device.

3. The device according to claim 1 wherein power supplied to the electrical component is supplied subsequent to termination of the radio frequency radiation.

4. The device according claim 1 wherein the electrical component is a transmitter.

5. The device according to claim 1 wherein the radio frequency radiation is in the range of 1 to 100 micrWB/mˆ2.

6. The device according to claim 1, wherein the operating switch is at least partially integral to a controller of the device.

7. The device according to claim 1 wherein the operating switch is to alter the power state of the device.

8. The device according to claim 1 comprising a coil, wherein the coil is to receive the radio frequency radiation.

9. The device according to claim 8 comprising a capacitor wherein the coil is in resonance with the capacitor.

10. The device according to claim 9 wherein the coil is an RF antenna.

11. The device according to claim 10 wherein the RF antenna is to receive control data.

12. The device according to claim 1 wherein the imager is a solid-state imager.

13. A method altering the power state of an ingestible imaging device, the method comprising:
irradiating the imaging device with an externally applied radio frequency radiation; and
in response to the irradiating, supplying power from a power source to an electrical component, wherein the power source and the electrical component are contained within the device.

14. The method according to claim 13 comprising positioning the device within a coil, the coil transmitting radio frequency radiation.

15. The method according to claim 13 comprising transmitting a signal to an RF operating switch to activate the device.

16. The method according to claim 13 comprising transmitting a signal to an RF operating switch to deactivate the device.

17. The method according to claim 13 wherein the electrical component is a transmitter, wherein the transmitter is to wirelessly transmit image data.

18. The method according to claim 13 comprising powering an electrical component subsequent to the irradiating.

19. The method according to claim 13 comprising:
capturing in-vivo image data; and
transmitting image data to an external receiver.

20. The method according to claim 13 comprising sensing an operation state of the device.

21. A system for activating an ingestible imaging device, the device comprising:
a mechanically static operating switch; and
a generator of radio frequency radiation suitable to activate the operating switch, the generator being external to the device.

22. The system according to claim 21 wherein the generator comprises a primary coil, and wherein the primary coil is to transmit the radio frequency radiation.

23. The system according to claim 21 wherein the device comprises a secondary coil, wherein the secondary coil is to receive the radio frequency radiation.

24. The system according to claim 21 wherein the generator of radio frequency radiation comprises a primary coil, and wherein the device is to be positioned within the primary coil.

25. The system according to claim 21 wherein the generator is to generate a first signal to activate the device and a second signal to deactivate the device.
